(19) United States
(12) Patent Application Publication
(10) Pub. No.: US 2025/0259400 A1
(43) Pub. Date: Aug. 14, 2025

(54) REAL-TIME ITEM RECOGNITION USING AUGMENTED REALITY

(71) Applicant: Omnicell, Inc., Mountain View, CA (US)

(72) Inventors: Prakash Pattisapu, Cary, NC (US); Jaime Jaramillo, Murfreesboro, TN (US); Aatish Sethi, Suwanee, GA (US); Rich Lau, Dix Hills, NY (US)

(73) Assignee: Omnicell, Inc., Mountain View, CA (US)

(21) Appl. No.: 18/439,528

(22) Filed: Feb. 12, 2024

(51) Int. Cl. G06T 19/00; G06Q 10/087; G06V 10/44

(52) U.S. Cl. CPC G06T 19/006; G06Q 10/087; G06V 10/44

(57) ABSTRACT

The present relates to real-time item recognition using augmented reality. This includes a method of item recognition using augmented reality. The method includes capturing a plurality of images with an augmented reality (AR) device, the plurality of images including an item, extracting with the AR device image features from the plurality of images, extracting with the AR device a plurality of words on the item from the plurality of images, identifying the item based on at least some of the image features and at least some of the plurality of words, accessing with the AR device at least one database of information relating to the item, determining to deliver the item based on the information relating to the item, and updating an inventory database upon confirmation of delivery of the item.

### BACKGROUND

The present generally relates to the field of item management and dispensing, and more specifically to the field of medication management and dispensation in healthcare facilities such as hospitals and pharmacies. More specifically, the present relates to the use of augmented reality (AR) to facilitate real-time recognition and verification of medications throughout the medication management process.

Retrieving a medication from a storage area or a storage device can be challenging for a user to quickly and reliably find an item such as a medication. This can relate to delays in providing a medication, increased costs, and risk of error in the form of the user selecting the wrong medication. Thus, improvements to item management, identification, and dispensing are desired.

### BRIEF SUMMARY

Aspects relate to real-time item recognition using augmented reality. One aspect relates to a method of item recognition using augmented reality. The method includes capturing a plurality of images with an augmented reality (AR) device, the plurality of images including an item, extracting with the AR device image features from the plurality of images, extracting with the AR device a plurality of words on the item from the plurality of images, identifying the item based on at least some of the image features and at least some of the plurality of words, accessing with the AR device at least one database of information relating to the item, determining to deliver the item based on the information relating to the item, and updating an inventory database upon confirmation of delivery of the item.

In some embodiments, the item can be a medication. In some embodiments, the item can be a medical device. In some embodiments, the item can be a consumable. In some embodiments, the item is stored in a medical storage area.

In some embodiments, the item is stored in a medical storage device. In some embodiments, the medical storage device can include a storage-device processor, and a storage-device memory. In some embodiments, the storage device memory can include an inventory database identifying items stored in the medical storage device, and a location in the medical storage device of the items stored in the medical storage device.

In some embodiments, the medical storage device comprises an automated dispensing cabinet (ADC). In some embodiments, the ADC can include a plurality of drawers, each of which drawers can include a plurality of compartments. In some embodiments, at least some of the items are stored each in a unique compartment. In some embodiments, the method further includes receiving identification of the item from a user of the AR device, determining a location of the item within the ADC, and indicating the item location to the user via the AR device.

In some embodiments, the at least one database of information relating to the item includes a medication database stored in memory accessible by the AR device via a communication network. In some embodiments, determining to deliver the item is further based on patient information. In some embodiments, the patient information is associated with a recipient of the item.

In some embodiments, the method includes retrieving the patient information from a patient database. In some embodiments, the patient database is stored in memory accessible by the AR device via the communication network. In some embodiments, determining to deliver the item based on the information relating to the item further includes determining that the item is not expired. In some embodiments, determining to deliver the item based on the information relating to the item further includes determining that a lot number of the item is not associated with a recall.

One aspect relates to a system for item recognition using augmented reality. The system includes a medical storage device. The medical storage device includes a storage-device processor, and a storage-device memory including an inventory database. The inventory database identifies items stored in the medical storage device and a location in the medical storage device of the items stored in the storage device. The system includes an augmented reality (AR) device communicatively coupled to the storage device. The AR device includes a camera, a display, a memory including computer executable instructions, and a processor. The processor can capture a plurality of images with the camera of the AR device, extract image features from the plurality of images, extract a plurality of words on the item from the plurality of images, identify the item based on at least some of the image features and at least some of the plurality of words, access at least one database of information relating to the item, determine to deliver the item based on the information relating to the item, receive a confirmation of delivery of the item from the storage device, and update the inventory database upon confirmation of delivery of the item. In some embodiments, the plurality of images can include an item.

In some embodiments, the item includes at least one of a medication, a medical device, or a consumable. In some embodiments, the medical storage device can be an automated dispensing cabinet (ADC). In some embodiments, the ADC can include a plurality of drawers, each of which drawers comprising a plurality of compartments, wherein at least some of the items are stored each in a unique compartment.

In some embodiments, the processor can further receive identification of the item from a user of the AR device, determine a location of the item within the ADC, and control the display to indicate the item location to the user. In some embodiments, determining the location of the item within the ADC can include querying the ADC for location information, and receiving the location information from the inventory database of the ADC. In some embodiments, the at least one database of information relating to the item includes a medication database stored in memory accessible by the AR device via a communication network. In some embodiments, determining to deliver the item is further based on patient information. In some embodiments, the patient information is associated with a recipient of the item.

In some embodiments, the processor can further retrieve the patient information from a patient database. In some embodiments, the patient database is stored in memory accessible by the AR device via the communication network. In some embodiments, determining to deliver the item based on the information relating to the item further includes determining that the item is not expired. In some embodiments, determining to deliver the item based on the information relating to the item further includes determining that a lot number of the item is not associated with a recall.

### BRIEF DESCRIPTION OF THE DRAWINGS

FIG. 1 is a graphical depiction of one embodiment of an AR device.

FIG. 2 is a schematic depiction of one embodiment of the AR device.

FIG. 3 is a schematic illustration of one embodiment of a system for real-time item recognition utilizing augmented reality.

FIG. 4 is a flowchart illustrating one embodiment of a process for item recognition utilizing an AR device.

FIG. 5 is a flowchart illustrating a first part of another embodiment of a process for item recognition utilizing an AR device.

FIG. 6 is a flowchart illustrating a second part of another embodiment of a process for item recognition utilizing an AR device.

FIG. 7 is a schematic depiction of a computer system that may be incorporated as part of the previously described computerized devices.

### DETAILED DESCRIPTION

Item management and distribution can be a key part of providing healthcare. For example, the providing of a medication to a patient include the storage and management of that medication before providing the medication to the patient, and the distribution of that medication to the patient. Similar management and distribution occur with other items in providing healthcare, including with medical devices, surgical implements, consumables, or the like.

Healthcare facilities frequently employ various storage and retrieval methods for managing items including medications. These can include but are not limited to automated medication dispensing cabinets (ADCs), vertical carousels, open shelves, and refrigerators. These devices can organize and manage the storage and dispensation of items including medications. Many of these devices include a grid of bins, each containing a specific item such as a specific medication. This layout can facilitate efficient and accurate retrieval of medications based on prescriptions or medication orders. In some embodiments, and to further aid in item retrieval, some devices can be equipped with a computer terminal with a screen display that indicates the bin location of a particular item such as a medication based on a column/row combination. In some embodiments, and with some automated devices such as cabinets and carousel, guiding lights can direct the user to the appropriate location for easier identification of the required medication's position. Depending on the size of the device compared to the conspicuousness of the guiding lights, even the guiding lights may be insufficient to assist a user in retrieving an item. These challenges increase as the size of the storage device increases and can be especially enhanced when the item is stored in a closed or in a warehouse-type situation.

While use of such devices have enhanced medication dispensation's accuracy and efficiency, these devices have limitations. For example, the reliance on visual matching and manual selection of medications can result in human errors, including selecting the wrong medication. Such errors can be particularly present in high-pressure or busy environments. These errors can have severe consequences, ranging from ineffective treatment to adverse drug events.

Conventional systems further increase the risk of user error as they force the user to continuously shift attention between the screen display and the bins while locating a medication, which can be time-consuming and mentally taxing, especially when dealing with a large number of medications.

The present provides improvement to item management and dispensing. Specifically, the present relates to the use of an augmented reality (AR) device and augmented reality to improve item management and dispensing. This can include use of the AR device and of AR to for item recognition, and specifically for real-time item recognition as part of item management and dispensing. As used herein, “real-time” refers to a process performed with a delay of no more than 200 ms, 250 ms, or 300 ms, or between 50 ms and 250 ms.

In some embodiments, all or portions of the item recognition can be performed on the AR device. This can include, for example, utilizing the AR device for: capturing one or several images and/or video; preprocessing the images and/or video; determining the validity of the images and/or video; extracting features from the images and/or video; performing optical character recognition (OCR) on the images and/or video, which OCR can include extracting one or several words from the images and/or video; and identifying the item based on the extracted features and/or extracted words. In some embodiments, the AR device can, in cooperation with the storage device and/or a memory, indicate a location of the item to the user, determine to deliver and/or dispense the item, and/or update an inventory when the item is delivered and/or dispensed. In some embodiments, this determination to dispense the item can be based on the extracted features and/or words, the item identification, and information retrieved from one or several databases including an item database and/or a patient database. In some embodiments, and based on information retrieved from these databases, the AR device can determine if the item selected for retrieval is the right item and/or is a good item or in other words is a non-expired item for which there are no recalls. In embodiments in which the item is a medication, the AR device can determine if the medication selected for retrieval is the right medication, the right medication dose, risks any reactions with others of the patient's medications, is a non-expired medication, and/or has no open recalls.

In some embodiments, a processor remote from the AR device can receive information from the AR device and can, in parallel and/or with a delay, evaluate the received information. In some embodiments, this can include: receiving the one or several images and/or video; preprocessing the images and/or video; determining the validity of the images and/or video; extracting features from the images and/or video; performing optical character recognition (OCR) on the images and/or video, which OCR can include extracting one or several words from the images and/or video; and identifying the item based on the extracted features and/or extracted words. In some embodiments, the processor remote from the AR device can perform the processing to confirm the processing of the AR device and to improve accuracy of processing.

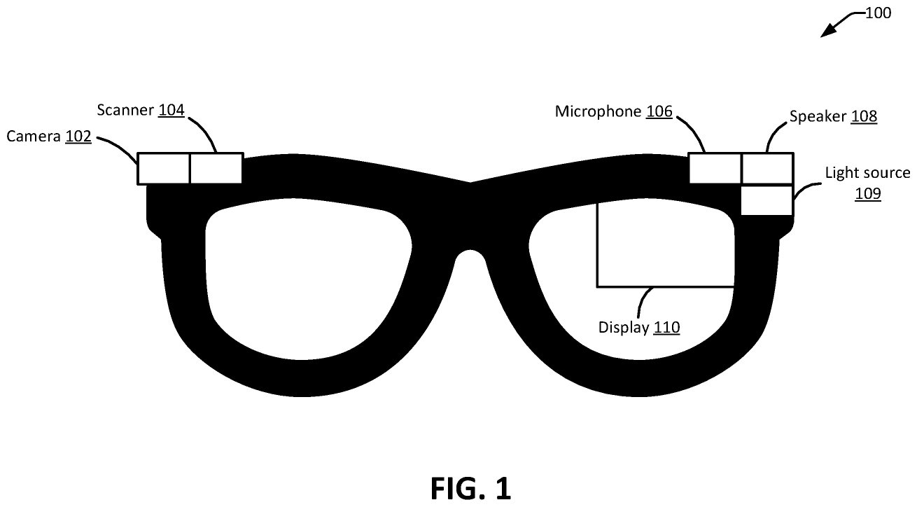

With reference now to FIG. 1, one embodiment of an AR device 100 is shown. The AR device 100 can include one or several sensors configured to receive information from the environment of the AR device 100 and one or several output features configured to provide information to the user of the AR device 100. The AR device can comprise a variety of form factors including, for example, goggles, glasses, monocle, helmet visor, headset, or the like.

The AR device 100 can, in some embodiments, comprise one or several cameras 102. The one or several cameras 102 can comprise one or several digital cameras configured to generate images and/or video embodied in digital data. In some embodiments, the one or several cameras 102 can be configured to continuously capture images of the user's field of view. In some embodiments, these cameras can include, for example, one or several standard cameras, one or several depth-sensing cameras, or the like.

The AR device 100 can include a scanner 104, also referred to herein as a reader 104. The scanner 104 can be configured to scan and/or read one or several computer readable codes such as, for example, one or several barcodes including one or several linear barcodes, one or several 2D barcodes, one or several matrix (2D) codes including, for example, one or several QR codes, or the like.

The AR device 100 can include one or several microphones 106. In some embodiments, the one or several microphones 106 can be configured to capture speaking by the user of the AR device 100 to thereby allow the user of the AR device 100 to control operation of the AR device 100. In some embodiments, the microphone 106 can be positioned proximate to the user's mouth, and/or can be positioned relatively more proximate to the user's mouth than other components of the AR device 100.

The AR device 100 can include a speaker 108. The speaker 108 can be configured to transform electrical signals into sounds perceptible by the user of the AR device 100. In some embodiments, the speaker 108 can be positioned proximate to the user's ear, can be oriented towards the user's ear, and/or can be positioned relatively more proximate to the user's ear than other components of the AR device 100.

The AR device 100 can include a light source 109. The light source 109 can, in some embodiments, be configured to illuminate the field of view of the AR device 100. In some embodiments, this illumination can facilitate capturing of images, and specifically can facilitate capturing high quality images.

The AR device 100 can include a display 110. The display 110 can be configured to transform and/or convert one or several electrical signals into one or several images, icons, text, words, or the like that can be seen by the user of the AR device 100. In some embodiments, the display can encompass all or portions of the field of view of the user of the AR device 100.

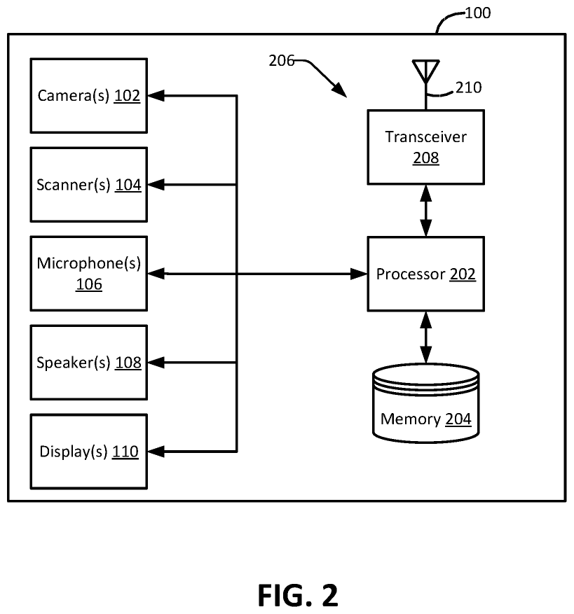

With reference now to FIG. 2, a schematic depiction of one embodiment of the AR device 100 is shown. As seen, and similar to FIG. 1, the AR device 100 can include one or several cameras 102, one or several scanners 104, one or several microphones 106, one or several speakers 108, and/or one or several displays 110. The AR device 100 can further include one or several processors 202. The one or several processors 202 can include one or several general-purpose processors including one or several computer chips, microprocessors, or the like. In some embodiments, the processors 202 can include one or several central processing units (CPUs), one or several graphics processing units (GPUs), or the like. In some embodiments, the one or several processors can comprise one or several special-purpose processors (such as digital signal processing chips, graphics acceleration processors, and/or the like.

The one or several processors 202 can be configured to communicate with the other components of the AR device 100. In some embodiments, this can include receiving information from the other components of the AR device 100 and generating one or several signals, including control signals, to control operation of the other components of the AR device 100. This can include, for example, controlling the camera 102 to capture one or several images, receiving the one or several images from the camera 102, and processing the one or several images. The processor 202 can further control the scanner 104 to scan one a computer readable code, and the processor 202 can read the machine-readable code. The processor 202 can control the microphone 106 to convert one or several sounds into electrical signals, which can be digital or analogue, and the processor 202 can receive the electrical signals for the microphone 106. The processor 202 can, in some embodiments, control the speaker 108 to generate one or several sounds. The processor 202 can, in some embodiments, control the display 110 to display one or several images, icons, text, words, or the like to the user.

The AR device 100 can further include a memory 204. In some embodiments, the memory 204 can be communicatively coupled with the processor 202. The memory 204 can any type of memory including, for example, primary memory, secondary memory, and/or cache memory. In some embodiments, the memory 204 can include volatile memory and/or non-volatile memory. The memory 204 can include stored computer code executable by the processor 202 to cause the processor to take one or several actions and/or to cause the processor to direct one or several components of the AR device 100 to take one or several actions.

The memory 204 can include one or several databases including, for example, an inventory database, a security database, an analysis database, or the like. In some embodiments, the AR device 100 can communicate with one or several other devices to receive information populating these databases and/or to receive these databases. In some embodiments, for example, the inventory database can include information identifying items in inventory in a medical storage device and/or in the medical storage area. In some embodiments, the inventory database can further include information identifying the location of those items in inventory in the medical storage device and/or the medical storage area. In some embodiments, information populating the inventory database can be received by the AR device 100 from the medical storage device and/or the medical storage area, and in some embodiments, the inventory database itself can be received by the AR device 100 from the medical storage device and/or the medical storage area.

In some embodiments, the security database can include information use for validating the user and such for validating the AR device 100 to the medical storage device and/or the medical storage area. In some embodiments, the security database can include a user identifier, password, one or several tokens, one or several keys including one or several private keys and/or public keys, or the like.

In some embodiments, the analysis database can include information and/or computer code for analyzing images captured by the AR device 100. In some embodiments, this information and/or computer code can include one or several models such as one or several machine learning models. In some embodiments, information and/or computer code in the analysis database can include information and/or computer code for performing preprocessing on images captured by the AR device 100, information and/or computer code for extracting one or several image features from the captured images, information and/or computer code for performing optical character recognition (OCR) on images captured by the AR device 100, information and/or computer code for performing text and/or word recognition and/or extraction images captured by the AR device 100, or the like.

The AR device 100 can include a communications module 206. In some embodiments, the communications module 206 can be communicatively coupled with the processor 202. The communications module 206 can, in some embodiments, be configured to allow the AR device 100 communicate with one or several devices such as, for example, with the medical storage device and/or medical storage area, and/or with one or several processors. The communications module 206 can communicate via one or several communications protocols, and specifically via one or several wireless communications protocols including, for example, Bluetooth, Wi-Fi, NFC, Zigbee, Z-wave, 6LoWPAN, cellular including 2G, 3G, 4G, 5G, or the like, LoRaWAN, LTE, or any other desired communications protocol.

The communications module 206 can, in some embodiments, include a transceiver 208 and an antenna 210. In some embodiments, the transceiver 208 can be configured to both transmit and communications and to receive communications. The transceiver 208 can be coupled to the antenna 210 which can be the interface between radio waves propagating through space and electrical currents used with the transceiver 208. In some embodiments, the transceiver 208 and/or the intent 210 can be configured for communication via the one or several communications protocols utilized by the communications module 206.

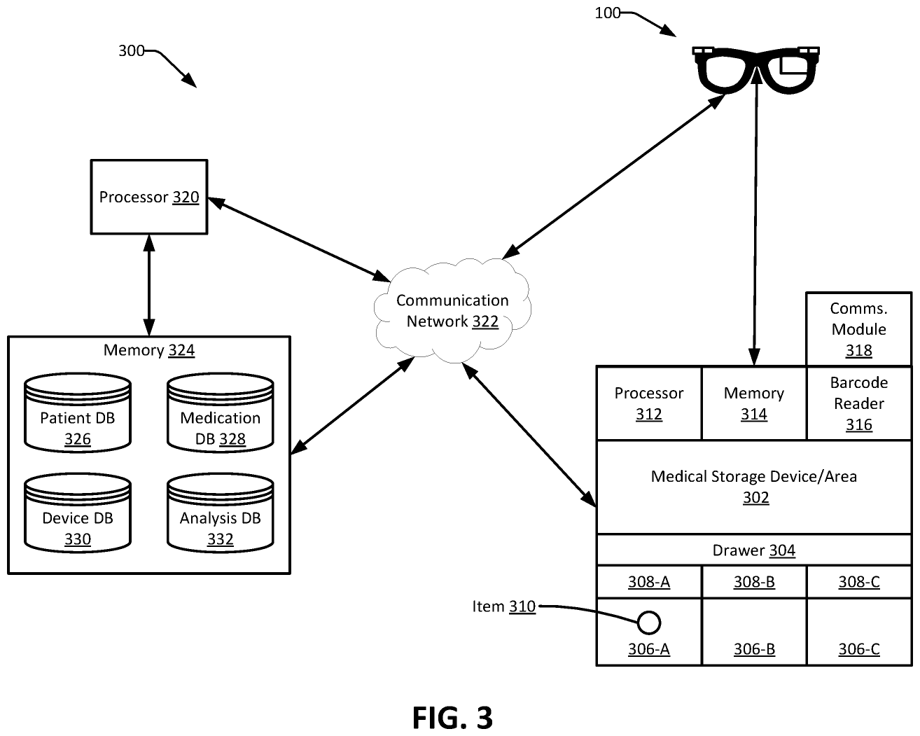

With reference now to FIG. 3, a schematic illustration of one embodiment of a system 300 for real-time item recognition utilizing augmented reality is shown. The system 300 can include the AR device 100. In some embodiments, the system 300 can further include a medical storage device 302 and/or medical storage area 302. The medical storage device 302 and/or medical storage area 302 can include a medical storage area and can be configured to store one or several items which items can include, for example, one or several medications, medical devices, and/or consumables such as bandages, sponges, gauze, tissues, or the like. In some embodiments, these medications can be in various forms including, for example, unit dose, blister packets, vials, and/or bottles. In some embodiments, the item packaging can each include identifiable information such as the item and/or medication name, an identifier such as the National Drug Code (NDC) identifier, the lot number, the serial number, the dosage, or the like. In some embodiments, some or all of this information can be used to identify the item.

The medical storage device 302 and/or medical storage area 302 can, in some embodiments, comprise one or several medication dispensing devices including one or several vertical carousels, open shells, refrigerators, automated medication dispensing cabinets (ADC), or the like. Embodiments of the medical storage device 302 and/or medical storage area 302, and/or additional types of dispensing units in which the invention may be embodied or which include features usable with embodiments of the invention are described in the following commonly owned U.S. patents and patent applications, the contents of which are hereby U.S. patents and patent applications, the contents of which are hereby incorporated by reference: U.S. Pat. No. 6,272,394, issued on Aug. 7, 2001 to Lipps, U.S. Pat. No. 6,385,505, issued on May 7, 2002 to Lipps, U.S. Pat. No. 6,760,643, issued on Jul. 6, 2004 to Lipps, U.S. Pat. No. 5,805,455, issued on Sep. 8, 1998 to Lipps, U.S. Pat. No. 6,609,047, issued on Aug. 19, 2003 to Lipps, U.S. Pat. No. 5,805,456, issued on Sep. 8, 1998 to Higham et al., U.S. Pat. No. 5,745,366, issued on Apr. 28, 1998 to Higham et al., an U.S. Pat. No. 5,905,653, issued on May 18, 1999 to Higham et al., U.S. Pat. No. 5,927,540, issued on Jul. 27, 1999 to Godlewski, U.S. Pat. No. 6,039,467, issued on Mar. 21, 2000 to Holmes, U.S. Pat. No. 6,640,159, issued on Oct. 28, 2003 to Holmes et al., U.S. Pat. No. 6,151,536, issued on Nov. 21, 2000 to Arnold et al., U.S. Pat. No. 5,377,864, issued on Jan. 3, 1995 to Blechl et al., U.S. Pat. No. 5,190,185, issued on Mar. 2, 1993 to Blechl, U.S. Pat. No. 6,975,922, issued on Dec. 13, 2005 to Duncan et al., U.S. Pat. No. 7,571,024, issued on Aug. 4, 2009 to Duncan et al., U.S. Pat. No. 7,835,819, issued on Nov. 16, 2010 to Duncan et al., U.S. Pat. No. 6,011,999, issued on Jan. 4, 2000 to Holmes, U.S. Pat. No. 7,348,884, issued on Mar. 25, 2008 to Higham, U.S. Pat. No. 7,675,421, issued on Mar. 9, 2010 to Higham, U.S. Pat. No. 6,170,929, issued on Jan. 9, 2001 to Wilson et al., U.S. Pat. No. 8,126,590, issued on Feb. 28, 2012 to Vahlberg et al., U.S. Pat. No. 8,280,550, issued in Oct. 2, 2012 to Levy et al., and U.S. Patent Application Publication No. 2012/0203377 of Paydar et al., published on Aug. 9, 2012.

In some embodiments, the medical storage device 302 and/or medical storage area 302 can be configured to organize and/or manage the storage and/or to dispensing of the one or several items which can include one or several medications, medical devices, and/or consumables. In some embodiments, the medical storage device 302 and/or medical storage area 302, and specifically ADC can include a plurality of drawers 304, each of which drawers 304 can include a plurality of compartments 306. In some embodiments, some or all these compartments can include a lid 308, which they can secure one or several items 310 in one or several compartments 306. In some embodiments, at least some of the items 310 are stored each in a unique compartment 306. Thus, as seen in FIG. 3, item 310 is stored in compartment 306-A and secured by lid 308-A.

The medical storage device 300 to can further include a processor 312, memory 314, a reader such as barcode reader 316, and/or a communications module 318. The processor 312, also referred to herein as a storage-device processor 312, can include one or several general-purpose processors including one or several computer chips, microprocessors, or the like. In some embodiments, the processor 312 can include one or several central processing units (CPUs), one or several graphics processing units (GPUs), or the like. In some embodiments, the processor 312 can comprise one or several special-purpose processors (such as digital signal processing chips, graphics acceleration processors, and/or the like.

The processor 312 can be configured to communicate with the other components of the medical storage device 302 and/or medical storage area 302. In some embodiments, this can include receiving information from the other components of the medical storage device 302 and/or medical storage area 302 and generating one or several signals, including control signals, to control operation of the other components of the medical storage device 302 and/or medical storage area 302. This can include, for example, controlling the barcode reader 316, controlling the communications module 318, and/or controlling one or more of the lids 308 to open, close, and/or lock.

The medical storage device 302 and/or medical storage area 302 can further include a memory 314, also referred to herein as a storage-device memory 314. In some embodiments, the memory 314 can be communicatively coupled with the processor 312. The memory 314 can any type of memory including, for example, primary memory, secondary memory, and/or cache memory. In some embodiments, the memory 314 can include volatile memory and/or non-volatile memory. The memory 314 can include stored computer code executable by the processor 312 to cause the processor to take one or several actions and/or to cause the processor to direct one or several components of the medical storage device 302 and/or medical storage area 302 to take one or several actions.

The memory 314 can include one or several databases including, for example, an inventory database, a security database, or the like. In some embodiments, for example, the inventory database can include information identifying items in inventory in the medical storage device 302 and/or medical storage area 302, or in other words, identifying items stored in the medical storage device 302 and/or medical storage area 302. In some embodiments, the inventory database can further include information identifying the location of those items in inventory in the medical storage device 302 and/or medical storage area 302.

In some embodiments, the security database can include information use for validating the user and such for validating the AR device 100 to the medical storage device 302 and/or medical storage area 302. In some embodiments, this security database can include information corresponding to information in the security database of the AR device 100. In some embodiments, the security database can include a user identifier, password, one or several tokens, one or several keys including one or several private keys and/or public keys, or the like.

The medical storage device 302 and/or medical storage area 302 can include a reader such as barcode reader 316. The barcode reader can, in some embodiments, be configured to read one or several machine-readable codes such as one or several barcodes on items being dispensed and/or to be dispensed from the medical storage device 302 and/or medical storage area 302. In some embodiments, the barcode reader 316 can be communicatively coupled with the processor 312 and can be controlled by the processor 312. In some embodiments this can include, for example, the processor 312 sending one or several signals to the barcode reader 316 directing the barcode reader 316 to read one or several machine-readable codes, and the barcode reader 316 providing one or several signals corresponding to the red one or several machine-readable codes to the processor 312.

The medical storage device 302 and/or medical storage area 302 can include a communications module 318. The communications module 318 can be communicatively coupled with the processor 312. The communications module 318 can, in some embodiments, be configured to allow the medical storage device 302 and/or medical storage area 302 to communicate with one or several devices such as, for example, with the AR device 100, and/or with one or several processors 320. The communications module 318 can communicate via one or several communications protocols, and specifically via one or several wireless communications protocols including, for example, Bluetooth, Wi-Fi, NFC, Zigbee, Z-wave, 6LoWPAN, cellular including 2G, 3G, 4G, 5G, or the like, LoRaWAN, LTE, or any other desired communications protocol.

In some embodiments, and as depicted in FIG. 3, the AR device 100 and the medical storage device 302 and/or medical storage area 302 can communicate directly with each other and/or can communicate via communication network 322. The communication network 322 can comprise a wired, wireless, or hybrid wired/wireless communication network 322. In some embodiments, the communication network 322 can include one or several local area networks (LAN), wide area networks (WAN), or the like. In some embodiments, the communication network 322 can include a public network including the internet, or can include a private network. Including, for example, a virtual private network.

The system 300 can include a processor 320. The processor 320 can include one or several processors, servers, computers, computing devices, or the like. In some embodiments, the processor 320 can be a processing service provided by, for example, a cloud service provider such as Amazon Web Services, Oracle, Microsoft Azure, or the like.

The processor 320 can be communicatively coupled with the other components of the system 300 either directly or via communications network 322. The processor 320 can, in some embodiments, receive signals from other components of the system 300 and can send on signals to the other components of the system 300. In some embodiments these signals sent by the processor 320 can include instructions directing and/or controlling aspects of operation of one or several of the other components of the system 300.

The system 300 can include memory 324, also referred to herein as central memory 324. The memory 324 can include physical memory or memory in the cloud. The memory 324 can include one or several instructions executable by the processor 320. These instructions, when executed by the processor 320 can cause the processor 320 to take one or several actions, some of which actions will be discussed at greater length below.

The memory 324 can include one or several databases 326-332 including, for example, a patient database 326, a medication database 328, a device database 330, and an analysis database 332. In some embodiments, the memory 324 and/or one or more of the databases 326-332 in the memory 324 is accessible by the AR device 100 and/or by the medical storage device 302 and/or medical storage area 302 via the communication network 322.

In some embodiments, the patient database 326 can include information relating to one or several patients. This information can include information identifying each of the one or several patients, and/or identifying one or several attributes of the one or several patients. In some embodiments, this information identifying one or several attributes the patient can identify, for example, patient age, height, weight, health conditions and/or health state, current medications, medication dosage, time of receipt of last medication, or the like.

The memory 324 can include the medication database 328. The medication database 328 can include information relating to one or several medications, and specifically relating to one or several medications stored in and/or distributed by the medical storage device 302 and/or medical storage area 302. This information can include, for example, information identifying one or several medication side effects, drug interactions, recalls, warnings, medical indications, dosing recommendations, or the like. In some embodiments, the medication database 328 in the memory 324 is accessible by the AR device 100 and/or by the medical storage device 302 and/or medical storage area 302 via the communication network 322.

The memory 324 can include the device database 330. The device database 330 can include information relating to one or several items which items can be one or several medical devices and/or consumables. In some embodiments, these one or several items can be stored in an/or distributed by the medical storage device 302 and/or medical storage area 302. This information relating to the items can include, for example, information identifying the items including information identifying one or several medical devices and/or consumables, information identifying one or several contraindications, recalls, warnings, medical indications, or the like. In some embodiments, the device database 330 in the memory 324 is accessible by the AR device 100 and/or by the medical storage device 302 and/or medical storage area 302 via the communication network 322.

The memory 324 can include the analysis database 332. The analysis database 332 can include information for use in analyzing images captured by the AR device 100. This information can include one or several machine learning models, one or several software's, or the like. In some embodiments, the analysis database 332 can include information, models, and/or software that when executed can extract image features from images captured by the AR device 100, can OCR the images captured by the AR device 100, can extract words on the item from the images captured by the AR device 100, can identify the item based on at least some of the image features and at least some of the words extracted from the images captured by the AR device 100, or the like. In some embodiments, the analysis database 332 in the memory 324 is accessible by the AR device 100 and/or by the medical storage device 302 and/or medical storage area 302 via the communication network 322.

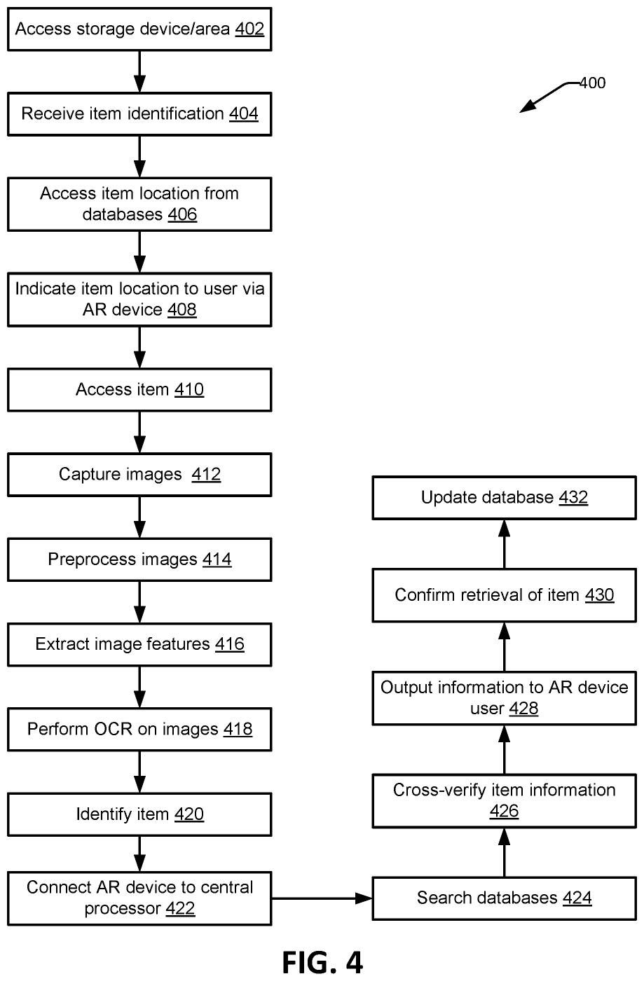

With reference now to FIG. 4, a flowchart illustrating one embodiment of a process 400 for item recognition utilizing an AR device 100. The process 400 can be performed by all or portions of the system 300 including by the AR device 100.

The process 400 begins at block 402, wherein the medical storage device 302 and/or medical storage area 302 is accessed. In some embodiments, this can include the user interacting with the medical storage device 302 and/or medical storage area 302 to ascertain the identity of the user and to determine whether the user is allowed access to the medical storage device 302 and/or medical storage area in some embodiments, this can be performed by the user directly interacting with medical storage device 302 and/or medical storage area, and in some embodiments, this can include the user interacting with medical storage device and/or medical storage area 302 via the AR device 100. In some embodiments, for example, the AR device 100 and medical storage device 302 and/or medical storage area 302 can utilize one or several user identifiers, passwords, tokens, keys, or the like according to a security protocol to establish the identity of the user and determine whether the user is allowed access to the medical storage device 302 and/or the medical storage area 302. In some embodiments, and after the identity and access of the user has been established, the user can access medical storage device 302 and/or the medical storage area 302. In some embodiments, this can include the user interacting with the storage-device processor 312 via the AR device 100.

At block 404 item identification is received. In some embodiments, the item identification can identify the item to be retrieved from the medical storage device 302 and/or the medical storage area 302. This item can be, in some embodiments, a medication, medical device, a consumable, and/or the like. In some embodiments, the item can be stored in the medical storage device 302 and/or in the medical storage area 302. In some embodiments, the item identification can be received by the AR device 100 from the user and/or from a computer system receiving inputs reflecting treatments to be provided to a patient, and in some embodiments, the item identification can be received by the medical storage device 302 and/or the medical storage area 300 to from the AR device 100.

At block 406 the item location within the medical storage device 302 and/or the medical storage area 302 is accessed in the inventory database in the memory 204. The location information can be accessed via communication between the AR device 100 and the medical storage device 302 and/or the medical storage area 302. In some embodiments, the AR device 100 can query the medical storage device 302 and/or the medical storage area 302, and specifically can query the memory 204 and/or the inventor database of the medical storage device 302 and/or the medical storage area 302 for location information for the item. In embodiments in which the medical storage device 302 and/or the medical storage area 302 is an ADC, this can include querying the ADC, and/or the memory 204 or inventory database of the ADC for location information.

In some embodiments, the location information can be received by the AR device 100 from the medical storage device 302 and/or the medical storage area 302. In some embodiments, this location information can be received by the AR device 100 via a communication with the medical storage device 302 and/or the medical storage area 302, which communication can be direct or indirect via the communication network 322.

At block 408, the item location is indicated to the user via the AR device 100. In some embodiments, this can include the processor 202 of the AR device 100 controlling the display 110 of the AR device 100 and/or the speaker 108 of the AR device 100 to indicate the location of the item in the medical storage device 302 and/or the medical storage area 302. In some embodiments, the AR device 100 can graphically indicate the location of the item in the medical storage device 302 and/or the medical storage area 302. This graphic indication can include providing the user via the display 110 one or several visual indicators of the location of the item in the medical storage device 302 and/or the medical storage area 302.

At block 410, the item can be accessed. In some embodiments, this can include, for example, manipulating a portion of the medical storage device 302 and/or the medical storage area 302 such that the item is visible. In some embodiments accessing the item can include, for example, opening the drawer 304 containing the item 310, opening the lid 308-A of the compartment 306-A containing the item 310, or the like.

At block 412 at least one image is captured by the AR device 100. In some embodiments, this can include capturing a plurality of images with the AR device 100, and specifically with the camera 102 of the AR device 100. In some embodiments, the at least one image can include the item 310. Likewise, in some embodiments, at least some of the plurality of images can include the item 310. In some embodiments some or all of these images can comprise video or can comprise still images.

At block 414, the captured image(s) are preprocessed. In some embodiments, this preprocessing can include modifying one or several attributes of some or all of the images such that the images comply with one or more image requirements. This can include, for example, modifying lighting, contrast, tilt, focus, or the like. In some embodiments, this pre-processing can be performed by the AR device 100, and specifically can be performed by the processor 202 of the AR device 100 according to computer code contained in the memory 204.

At block 416 one or more image features are extracted from at least one of the image(s). In some embodiments, these image features can include one or more features of the item and/or item packaging. This can include, for example, features relating to the size, shape, and/or boundaries of all or portions of the item such as, for example, the size, shape, text, logos, distinctive packaging elements, and/or boundaries of a label of the item. In some embodiments, these features can further include a color, a texture, or the like. In some embodiments, the image features can be extracted by the AR device 100, and specifically by the processor 202 of the AR device 100. In some embodiments, these image features can be extracted according to computer code contained in the memory.

In some embodiments, these image features can be extracted according to computer code contained in the memory 204 of the AR device 100, which computer code can be received from the central memory 324 and specifically from the analysis database 332 of the central memory 324. In some embodiments, the AR device 100 can receive periodic updates from the central processor 320 and specifically from the analysis database 332 of the computer code for extracting the image features from the images.

In some embodiments, image features can be extracted by the central processor 320 according to computer code contained in the memory 324 and specifically in the analysis database 332. In some embodiments, the extraction of image features by the central processor 320 can be performed in addition to the extraction of image features by the AR device 100. In some embodiments, this extraction of image features by the central processor 320 can be performed to validate and/or check the image features extracted by the AR device 100.

At block 418 optical character recognition (OCR) is performed on some or all of the captured images. In some embodiments, this can include performing OCR on portions of the images containing the item. In some embodiments, the portions of the images containing the item can be identified based on the extracted image features.

In some embodiments, performing the OCR on all or portions of some or all of the images can include extracting with the AR device 100 one or several words on the item from some or all of the images. In some embodiments, this extraction can include the evaluation of the extracted words to identify one or several key words, such as words identifying the item and attributes of the item such as the dosage of a medication, quantity of medication, item lot number, item serial number, or the like.

In some embodiments, the OCR and/or word extraction can be performed according to computer code contained in the memory 204 of the AR device 100, which computer code can be received from the central memory 324 and specifically from the analysis database 332 of the central memory 324. In some embodiments, the AR device 100 can receive periodic updates from the central processor 320 and specifically from the analysis database 332 of the computer code for extracting the image features from the images.

In some embodiments, the OCR and/or word extraction can be performed by the central processor 320 according to computer code contained in the memory 324 and specifically in the analysis database 332. In some embodiments, the OCR and/or word extraction by the central processor 320 can be performed in addition to the OCR and/or word extraction by the AR device 100. In some embodiments, this OCR and/or word extraction by the central processor 320 can be performed to validate and/or check the OCR and/or word extraction by the AR device 100.

At 420, the item is identified. In some embodiments, the item can be identified by the AR device 100, and specifically by the processor 202 of the AR device 100. The item can, in some embodiments, be identified based on at least one or more of the image features and/or on at least one or more of the words extracted at step 418. In some embodiments, the item can be identified by ingesting some or all of the image features, extracted characters, and/or extracted text into a machine learning model. In some embodiments, the machine learning model can be configured to generate a prediction of the identification of the item based on these ingested inputs. In some embodiments, the item can be identified via a pattern recognition technique, which technique can identify recurring patterns and/or features and thereby identify the item. In some embodiments, these recurring patterns and/or features can be inputs ingested into the machine learning model, which machine learning model generates a prediction of the identification of the item.

At bock 422, the AR device 100 connects to the central processor 320. In some embodiments, this connection can be via the communication network 322. In some embodiments, this connection can be a secure connection. In some embodiments, this secure connection can be with the central processor 320 and/or with the memory 324 and specifically with one or more of the databases 326-332 in the memory 324. In some embodiments, this connection can be secure and can comply with healthcare regulations such as regulations to protect patient information.

At block 424, the AR device 100 searches and/or queries the central memory 324 for information relevant to item. In some embodiments, these queries can be based on, for example, the item name, and one or several item attributes. In embodiments in which the item is a medication, this information can include the medication name, dosage, or the like. In some embodiments, this query can be based on, for example, the item name, NDC, lot number, dosage, shape, color, size, or the like. In some embodiments, this can include the AR device 100 accessing at least one database of information relating to the item. In some embodiments, this can include searching and/or querying the one or more of the patient database 326, the medication database 328, and the device database 330. In some embodiments, the AR device 100 searches and/or queries the central memory 324 based on information relating to the item and the patient receiving the item.

For example, in some embodiments, the AR device 100 can search and/or query the patient database 326 for information relating to the patient and relevant to providing the item to the patient. This can include, for example, searching and/or querying the patient database 326 for information relating to current medications taken by the patient. This can include, for example, information identifying current medications taken by the patient, dosages of medications taken by the patient, when a medication was last taken by the patient, or the like. In some embodiments, the AR device 100 can further search and/or query the patient database 100 for information indicating one or several attributes of the patient such as one or several allergies.

In some embodiments, and in response to the search and/or queries of the patient database 326, the AR device 100 can receive information relevant to the patient.

The AR device 100 can further search and/or query the medication database 328 and/or the device database 330 for information relevant to the item. This information can include, for example, adverse effects, medical indications treated by the item, risks, expiration dates, recalls, or the like. In some embodiments, this information can be linked with one or several attributes of the item such as, for example, one or several serial numbers, lot numbers, or the like.

In some embodiments, and in response to the search and/or queries of the medication database 328 and/or the device database 330, the AR device 100 can receive information relevant to the item.

At block 426, the AR device 100 cross-verifies item information. This includes evaluating the item based on the information retrieved and/or received in block 424. In some embodiments, this can include determining with the AR device 100 whether to deliver and/or dispense the item to the user based on the information relating to the item and/or relating to the patient. In some embodiments, this patient information can be associated with the recipient of the item. In some embodiments, determining whether to deliver the item can include determining whether the patient is allergic to the item, whether the patient takes a medication that could adversely interact with the item, whether the item is expired, whether a recall is associated with the item and specifically whether a recall is associated with the item serial number and/or lot number, or the like.

In some embodiments, the cross-verification can include that the item imaged by the AR device 100 matches the item in the inventory database of the medical storage device 302 and/or the medical storage area 302. In some embodiments, this cross-verification can prevent the dispensing and/or delivery of the wrong item.

At block 428, information is output by the AR device 100 to the user of the AR device 100. This information can include patient information, item information, and/or information relating to the determination of whether to deliver and/or dispense the item. In some embodiments, this information can include item name and/or item attributes, and specifically can include medication name, dosage, administration instructions, potential side effects, or the like. In some embodiments, this information can further indicate expiration status of the medication such as if the item and/or medication is expired or is about to expire, when a needed item and/or medication is out of stock or are low in stock, information relating to any recall relevant to the item and/or medication including identification of any relevant recall by lot number, indication of any potential counter reactions when administered with other medications, or the like. In some embodiments, this information can be displayed to the user next to the item in the user's field of view. In some embodiments, this information can include patient information such as the patient's name, allergies, other prescribed medications, or the like. In some embodiments, patient information can be provided to the user of the AR device 100 adjacent to item information in the user's field of view.

In some embodiments, this information can be provided via the display 110 and/or via the speaker 108 of the AR device. The providing of this information can include the display of the item information to the user of the AR device 100 via the display 110, and specifically via the overlaying of the item information onto the item and/or adjacent to the item as viewed by the user of the AR device 100.

In some embodiments, the user can be further provided with one or several virtual interactive elements. This can include an interactive element, the virtual manipulation of which causes the AR device 100 to provide the user of the AR device 100 further information such as further information relating to the item. In some embodiments, manipulation of the virtual element can serve as an indicator of completion of an action by the user such as, for example, as an indicator of completion of dispensing and/or delivery of the item.

At block 430, the AR device 100 can confirm retrieval of the item for example, item. In some embodiments, this can include receiving information from the medical storage device 302 and/or medical storage area 302 indicating that the item has been retrieved, delivered, and/or dispensed.

At block 432, the inventory database of the medical storage device 302 and/or medical storage area 302 is updated to reflect the retrieval, delivery, and/or dispensing of the item. In some embodiments, the updating of the inventory database can be performed subsequent to receipt of information and/or a command from the AR device 100 directing the updating of the inventory database.

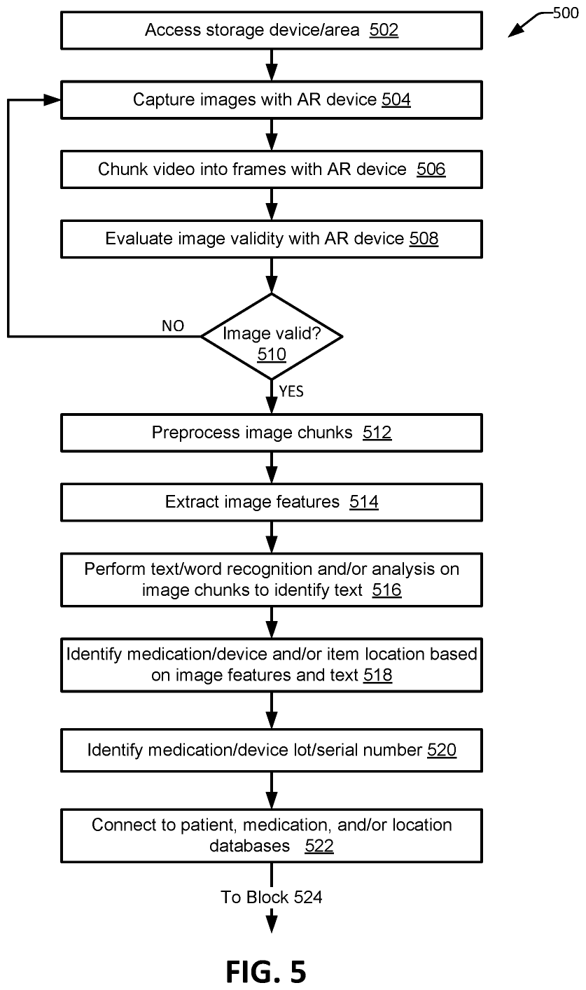

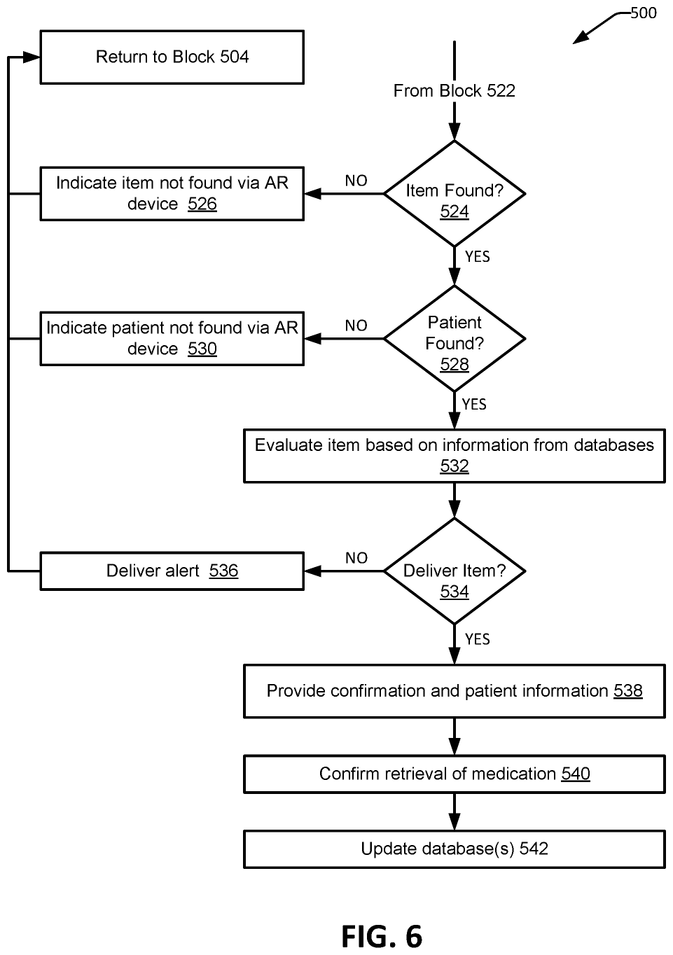

With reference now to FIGS. 5 and 6, a flowchart illustrating another embodiment of a process 500 for item recognition utilizing an AR device 100 is shown. The process 500 can be performed by all or portions of the system 300 including by the AR device 100.

The process 500 begins at block 502, wherein the medical storage device 302 and/or medical storage area 302 is accessed. In some embodiments, this can include the user interacting with the medical storage device 302 and/or medical storage area 302 to ascertain the identity of the user and to determine whether the user is allowed access to the medical storage device 302 and/or medical storage area in some embodiments, this can be performed by the user directly interacting with medical storage device 302 and/or medical storage area, and in some embodiments, this can include the user interacting with medical storage device and/or medical storage area 302 via the AR device 100. In some embodiments, for example, the AR device 100 and medical storage device 302 and/or medical storage area 302 can utilize one or several user identifiers, passwords, tokens, keys, or the like according to a security protocol to establish the identity of the user and determine whether the user is allowed access to the medical storage device 302 and/or the medical storage area 302. In some embodiments, and after the identity and access of the user has been established, the user can access medical storage device 302 and/or the medical storage area 302. In some embodiments, this can include the user interacting with the storage-device processor 312 via the AR device 100.

At block 504, at least one image is captured by the AR device 100. In some embodiments, this can include capturing a plurality of images with the AR device 100, and specifically with the camera 102 of the AR device 100. In some embodiments, the at least one image can include the item 310. Likewise, in some embodiments, at least some of the plurality of images can include the item 310. In some embodiments some or all of these images can comprise video or can comprise still images.

At block 506 the images, specifically when the images comprise video, can be chunked into frames via the AR device 100. In some embodiments, this can include dividing the video into frames.

At block 508 validity of the captured images is evaluated by the AR device 100. In some embodiments, this can include determining whether the images meet specific minimum requirements for usability. This can include, for example, having sufficient focus, lighting, resolution, or the like. In some embodiments, for example, the user can too quickly shift the direction of their gaze, thereby adversely impacting quality of captured images. In some embodiments, the determination of the validity of the captured images can include determining whether the user of the AR device 100 maintained a sufficiently constant gaze so as to allow use of captured images.

At block 510, it is determined if the captured images are valid. In some embodiments, this determination can be made based on the evaluation of block 508. If it is determined that the image is not valid, then the process can terminate or can return to block 504 and proceed as outlined above.

If it is determined that the image is valid, then the process 500 proceeds to block 512, wherein the image chunks are preprocessed. In some embodiments, this preprocessing can include modifying one or several attributes of some or all of the images such that the images comply with one or more image requirements. This can include, for example, modifying lighting, contrast, tilt, focus, noise reduction cropping, or the like. In some embodiments, this pre-processing can be performed by the AR device 100, and specifically can be performed by the processor 202 of the AR device 100 according to computer code contained in the memory 204.

At block 514 one or more image features are extracted from at least one of the image(s). The one or more image features can be extracted by the AR device 100 and/or by the processor 320. In some embodiments, the extraction of the one or more image features can be the same as performed as a part of block 416 discussed above.

At block 516, text and/or word recognition and/or analysis is performed on image chunks to identify text. In some embodiments, the step of block 516 can be performed by the AR device 100 and/or the processor 320. In some embodiments, the step of block 516 can be the same as the step of block 418 described above.

At block 518 the item and/or the location of the item is identified. In some embodiments, the item and/or the location of the item can be identified by the AR device 100 and/or the processor 320. In some embodiments, the item and/or the location of the item can be identified by the AR device 100 working in combination with the medical storage device 302 and/or the medical storage area 302. In some embodiments, the item can be identified based on at least one or more of the image features and/or on at least one or more of the extracted words.

In some embodiments, the location of the item can be determined by the AR device 100 alone or in combination with the medical storage device 302 and/or the medical storage area 302. In some embodiments, the AR device 100 can, based on the identification of the item, query the medical storage device 302 and/or the medical storage area 302 or the location of the item. The medical storage device 302 and/or the medical storage area 302 can, in some embodiments, provide this location information to the AR device 100.

At block 520, the item lot number and/or serial number are determined. In some embodiments, this determination can be made by the AR device 100. In some embodiments, this determination can be made based on the extracted text and/or words. In some embodiments, for example, the AR device 100 can identify the lot number and/or the serial number from the words extracted from the images.

At block 522, the AR device 100 can connect to the memory 324 via, for example, the communication network 322. In some embodiments, the AR device 100 can connect to one or more of the patient database 326, the medication database 328, and/or the device database 330 via the communication network 322. In some embodiments, this can include the AR device 100 querying one or more of the databases 326, 328, 330 for information and receiving this information from one or more of the databases 326, 328, 330.

At block 524, it is determined by the AR device 100 and based on information received from the one or more databases 328, 330 if the item is found in the databases 328, 330. If the item is not found in the databases 328, 330, then the process 500 proceeds to block 526, and the AR device 100 indicates to the user that the item was not found in the databases 328, 330. The process 500 then can terminate, or can proceed to block 504 and proceed as outlined above. Alternatively, if it is determined that the item is found in the databases 328, 330, then the process 500 can proceed to the next step.

At step 528, it is determined by the AR device 100 and based on information received from the patient database 326 if the patient is found in the patient database 326. If the patient is not found in the patient database 326, then the process 500 proceeds to block 530, and the AR device 100 indicates to the user that the patient was not found in the patient database 326. The process 500 then can terminate, or can proceed to block 504 and proceed as outlined above. Alternatively, if it is determined that the patient is found in the patient database 326, then the process 500 can proceed to the next step.

At block 534, the item is evaluated by the AR device 100 based on information received from the databases 326, 328, 330. In some embodiments, this can include determining whether the patient is allergic to the item, whether the patient takes a medication that could adversely interact with the item, whether the item is expired, whether a recall is associated with the item and specifically whether a recall is associated with the item serial number and/or lot number, or the like.

At block 534, it is determined whether to deliver and/or dispense the item. In some embodiments, the determination of block 534 can be performed by the AR device 100 and/or by the medical storage device 302 and/or the medical storage area 302. This determination can, in some embodiments, be made based on the evaluation of block 532. If it is determined to not deliver and/or dispense the item, then the process 500 can proceed to block 536 and can provide an alert via the AR device 100 to the user indicating that the item will not be delivered and/or dispensed and/or providing a recommendation to not deliver and/or dispense the item. In some embodiments, this can include providing a graphical indicator, such as a red cross over the item in the field of view of the user of the AR device 100. In some embodiments, the alert can further include reasons for the determination to not deliver and/or dispense the item and/or for the recommendation to not deliver and/or dispense the item. After the delivery of the alert to the user, the process 500 can terminate or can return to block 504 and proceed as outlined above.

Alternatively, if it is determined to deliver and/or dispense the item, the process 500 can proceed to block 538, wherein item confirmation and/or patient information can be provided by the AR device 100 to the user. In some embodiments, the item confirmation can comprise an indication of the determination of block 534 to deliver and/or dispense the item. This confirmation can comprise a graphical indication of the confirmation, such as a green check mark. In some embodiments, this can further include information identifying one or several steps and/or actions to take to dispense and/or deliver the item. In some embodiments, this information can include identification of a location of the item in the medical storage device 302 and/or the medical storage area 302.

At block 540, a confirmation of retrieval of the item can be received by the AR device 100. In some embodiments, this can comprise a confirmation from the medical storage device 302 and/or the medical storage area 302 that the item has been dispensed and/or delivered.

At block 542, one or several databases are updated. This can include updating one or several databases by the medical storage device 302 and/or the medical storage area 302, updating one or several databases by the processor 320, and/or updating one or several databases by the medical storage device 302 and/or the medical storage area 302. In some embodiments, the AR device 100 can update the inventor database stored on the AR device 100, the medical storage device 302 and/or the medical storage area 302 can update the inventory database stored in the memory 314 of the medical storage device 302 and/or the medical storage area 302, and/or the processor 320 can update the patient database 326 to indicate the dispensing of the item for the patient.

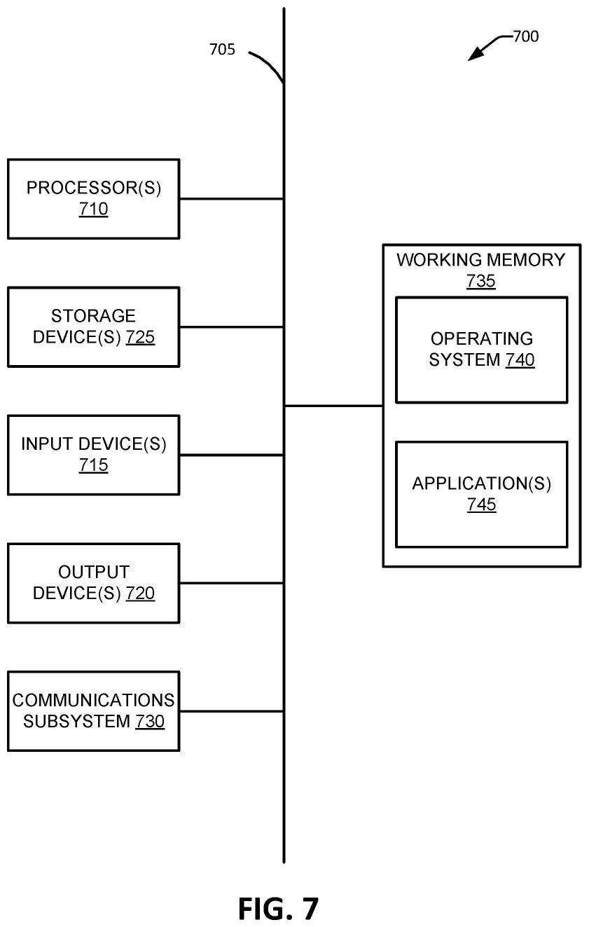

With reference now to FIG. 7, a computer system may be incorporated as part of the previously described computerized devices. For example, computer system 700 can represent some of the components of systems, components, and/or other computing devices described herein. FIG. 7 provides a schematic illustration of one embodiment of a computer system 700 that can perform the methods provided by various other embodiments, as described herein. FIG. 7 is meant only to provide a generalized illustration of various components, any or all of which may be utilized as appropriate. FIG. 7, therefore, broadly illustrates how individual system elements may be implemented in a relatively separated or relatively more integrated manner.

The computer system 700 is shown comprising hardware elements that can be electrically coupled via a bus 705 (or may otherwise be in communication, as appropriate). The hardware elements may include a processing unit 710, including without limitation one or more processors, such as one or more special-purpose processors (such as digital signal processing chips, graphics acceleration processors, and/or the like); one or more input devices 715, which can include without limitation a keyboard, a touchscreen, receiver, a motion sensor, an imaging device, and/or the like; and one or more output devices 720, which can include without limitation a display device, a speaker, and/or the like.

The computer system 700 may further include (and/or be in communication with) one or more non-transitory storage devices 725, which can comprise, without limitation, local and/or network accessible storage, and/or can include, without limitation, a disk drive, a drive array, an optical storage device, a solid-state storage device such as a random access memory (“RAM”) and/or a read-only memory (“ROM”), which can be programmable, flash-updateable and/or the like. Such storage devices may be configured to implement any appropriate data stores, including without limitation, various file systems, database structures, and/or the like.

The computer system 700 might also include a communication interface 730, which can include without limitation a modem, a network card (wireless or wired), an infrared communication device, a wireless communication device and/or chipset (such as a Bluetooth™ device, a 502.11 device, a Wi-Fi device, a WiMAX device, an NFC device, cellular communication facilities, etc.), and/or similar communication interfaces. The communication interface 730 may permit data to be exchanged with a network (such as the network described below, to name one example), other computer systems, and/or any other devices described herein. In many embodiments, the computer system 700 will further comprise a non-transitory working memory 735, which can include a RAM or ROM device, as described above.

The computer system 700 also can comprise software elements, shown as being currently located within the working memory 735, including an operating system 740, device drivers, executable libraries, and/or other code, such as one or more application programs 745, which may comprise computer programs provided by various embodiments, and/or may be designed to implement methods, and/or configure systems, provided by other embodiments, as described herein. Merely by way of example, one or more procedures described with respect to the method(s) discussed above might be implemented as code and/or instructions executable by a computer (and/or a processor within a computer); in an aspect, then, such special/specific purpose code and/or instructions can be used to configure and/or adapt a computing device to a special purpose computer that is configured to perform one or more operations in accordance with the described methods.

A set of these instructions and/or code might be stored on a computer-readable storage medium, such as the storage device(s) 725 described above. In some cases, the storage medium might be incorporated within a computer system, such as computer system 700. In other embodiments, the storage medium might be separate from a computer system (e.g., a removable medium, such as a compact disc), and/or provided in an installation package, such that the storage medium can be used to program, configure and/or adapt a special purpose computer with the instructions/code stored thereon. These instructions might take the form of executable code, which is executable by the computer system 700 and/or might take the form of source and/or installable code, which, upon compilation and/or installation on the computer system 700 (e.g., using any of a variety of available compilers, installation programs, compression/decompression utilities, etc.) then takes the form of executable code.

Substantial variations may be made in accordance with specific requirements. For example, customized hardware might also be used, and/or particular elements might be implemented in hardware, software (including portable software, such as applets, etc.), or both. Moreover, hardware and/or software components that provide certain functionality can comprise a dedicated system (having specialized components) or may be part of a more generic system. For example, a risk management engine configured to provide some or all of the features described herein relating to the risk profiling and/or distribution can comprise hardware and/or software that is specialized (e.g., an application-specific integrated circuit (ASIC), a software method, etc.) or generic (e.g., processing unit 710, applications 745, etc.) Further, connection to other computing devices such as network input/output devices may be employed.

Some embodiments may employ a computer system (such as the computer system 700) to perform methods in accordance with the disclosure. For example, some or all of the procedures of the described methods may be performed by the computer system 700 in response to processing unit 710 executing one or more sequences of one or more instructions (which might be incorporated into the operating system 740 and/or other code, such as an application program 745) contained in the working memory 735. Such instructions may be read into the working memory 735 from another computer-readable medium, such as one or more of the storage device(s) 725. Merely by way of example, execution of the sequences of instructions contained in the working memory 735 might cause the processing unit 710 to perform one or more procedures of the methods described herein.

The terms “machine-readable medium” and “computer-readable medium,” as used herein, refer to any medium that participates in providing data that causes a machine to operate in a specific fashion. In an embodiment implemented using the computer system 700, various computer-readable media might be involved in providing instructions/code to processing unit 710 for execution and/or might be used to store and/or carry such instructions/code (e.g., as signals). In many implementations, a computer-readable medium is a physical and/or tangible storage medium. Such a medium may take many forms, including but not limited to, non-volatile media, volatile media, and transmission media. Non-volatile media include, for example, optical and/or magnetic disks, such as the storage device(s) 725. Volatile media include, without limitation, dynamic memory, such as the working memory 735. Transmission media include, without limitation, coaxial cables, copper wire, and fiber optics, including the wires that comprise the bus 705, as well as the various components of the communication interface 730 (and/or the media by which the communication interface 730 provides communication with other devices). Hence, transmission media can also take the form of waves (including without limitation radio, acoustic and/or light waves, such as those generated during radio-wave and infrared data communications).

Common forms of physical and/or tangible computer-readable media include, for example, a magnetic medium, optical medium, or any other physical medium with patterns of holes, a RAM, a PROM, EPROM, a FLASH-EPROM, any other memory chip or cartridge, a carrier wave as described hereinafter, or any other medium from which a computer can read instructions and/or code.

The communication interface 730 (and/or components thereof) generally will receive the signals, and the bus 705 then might carry the signals (and/or the data, instructions, etc. carried by the signals) to the working memory 735, from which the processor(s) 705 retrieves and executes the instructions. The instructions received by the working memory 735 may optionally be stored on a non-transitory storage device 725 either before or after execution by the processing unit 710.

The methods, systems, and devices discussed above are examples. Some embodiments were described as processes depicted as flow diagrams or block diagrams. Although each may describe the operations as a sequential process, many of the operations can be performed in parallel or concurrently. In addition, the order of the operations may be rearranged. A process may have additional steps not included in the figure. Furthermore, embodiments of the methods may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium. Processors may perform the associated tasks.

It should be noted that the systems and devices discussed above are intended merely to be examples. It must be stressed that various embodiments may omit, substitute, or add various procedures or components as appropriate. Also, features described with respect to certain embodiments may be combined in various other embodiments. Different aspects and elements of the embodiments may be combined in a similar manner. Also, it should be emphasized that technology evolves and, thus, many of the elements are examples and should not be interpreted to limit the scope of the invention.

Specific details are given in the description to provide a thorough understanding of the embodiments. However, it will be understood by one of ordinary skill in the art that the embodiments may be practiced without these specific details. For example, well-known structures and techniques have been shown without unnecessary detail in order to avoid obscuring the embodiments. This description provides example embodiments only, and is not intended to limit the scope, applicability, or configuration of the invention. Rather, the preceding description of the embodiments will provide those skilled in the art with an enabling description for implementing embodiments of the invention. Various changes may be made in the function and arrangement of elements without departing from the spirit and scope of the invention.

The methods, systems, devices, graphs, and tables discussed above are examples. Various configurations may omit, substitute, or add various procedures or components as appropriate. For instance, in alternative configurations, the methods may be performed in an order different from that described, and/or various stages may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims. Additionally, the techniques discussed herein may provide differing results with different types of context awareness classifiers.

While illustrative and presently preferred embodiments of the disclosed systems, methods, and machine-readable media have been described in detail herein, it is to be understood that the inventive concepts may be otherwise variously embodied and employed, and that the appended claims are intended to be construed to include such variations, except as limited by the prior art.

Unless defined otherwise, all technical and scientific terms used herein have the same meaning as commonly or conventionally understood. As used herein, the articles “a” and “an” refer to one or to more than one (i.e., to at least one) of the grammatical object of the article. By way of example, “an element” means one element or more than one element. “About” and/or “approximately” as used herein when referring to a measurable value such as an amount, a temporal duration, and the like, encompasses variations of ±20% or ±10%, ±5%, or ±0.1% from the specified value, as such variations are appropriate to in the context of the systems, devices, circuits, methods, and other implementations described herein. “Substantially” as used herein when referring to a measurable value such as an amount, a temporal duration, a physical attribute (such as frequency), and the like, also encompasses variations of ±20% or ±10%, ±5%, or ±0.1% from the specified value, as such variations are appropriate to in the context of the systems, devices, circuits, methods, and other implementations described herein. As used herein, including in the claims, “and” as used in a list of items prefaced by “at least one of” or “one or more of” indicates that any combination of the listed items may be used. For example, a list of “at least one of A, B, and C” includes any of the combinations A or B or C or AB or AC or BC and/or ABC (i.e., A and B and C). Furthermore, to the extent more than one occurrence or use of the items A, B, or C is possible, multiple uses of A, B, and/or C may form part of the contemplated combinations. For example, a list of “at least one of A, B, and C” may also include AA, AAB, AAA, BB, etc.

Having described several embodiments, it will be recognized by those of skill in the art that various modifications, alternative constructions, and equivalents may be used without departing from the spirit of the invention. For example, the above elements may merely be a component of a larger system, wherein other rules may take precedence over or otherwise modify the application of the invention. Also, a number of steps may be undertaken before, during, or after the above elements are considered. Accordingly, the above description should not be taken as limiting the scope of the invention.

Also, the words “comprise”, “comprising”, “contains”, “containing”, “include”, “including”, and “includes”, when used in this specification and in the following claims, are intended to specify the presence of stated features, integers, components, or steps, but they do not preclude the presence or addition of one or more other features, integers, components, steps, acts, or groups.

What is claimed is:

1. A method of item recognition using augmented reality, the method comprising:
capturing a plurality of images with an augmented reality (AR) device, the plurality of images including an item;
extracting with the AR device image features from the plurality of images;
extracting with the AR device a plurality of words on the item from the plurality of images;
identifying the item based on at least some of the image features and at least some of the plurality of words;
accessing with the AR device at least one database of information relating to the item;
determining to deliver the item based on the information relating to the item; and
updating an inventory database upon confirmation of delivery of the item.

2. The method of claim 1, wherein the item comprises a medication.

3. The method of claim 1, wherein the item comprises a medical device.

4. The method of claim 1, wherein the item comprises a consumable.

5. The method of claim 1, wherein the item is stored in a medical storage area.

6. The method of claim 1, wherein the item is stored in a medical storage device.

7. The method of claim 6, wherein the medical storage device comprises:
a storage-device processor; and
a storage-device memory, the storage device memory comprising an inventory database identifying:
items stored in the medical storage device; and
a location in the medical storage device of the items stored in the medical storage device.

8. The method of claim 7, wherein the medical storage device comprises an automated dispensing cabinet (ADC).

9. The method of claim 8, wherein the ADC comprises a plurality of drawers, each of which drawers comprising a plurality of compartments, wherein at least some of the items are stored each in a unique compartment.

10. The method of claim 9, further comprising:
receiving identification of the item from a user of the AR device;
determining a location of the item within the ADC; and
indicating the item location to the user via the AR device.

11. The method of claim 1, wherein the at least one database of information relating to the item comprises a medication database stored in memory accessible by the AR device via a communication network.

12. The method of claim 11, wherein determining to deliver the item is further based on patient information, wherein the patient information is associated with a recipient of the item.

13. The method of claim 12, further comprising retrieving the patient information from a patient database.

14. The method of claim 13, wherein the patient database is stored in memory accessible by the AR device via the communication network.

15. The method of claim 14, wherein determining to deliver the item based on the information relating to the item further comprises determining that the item is not expired.

16. The method of claim 15, wherein determining to deliver the item based on the information relating to the item further comprises determining that a lot number of the item is not associated with a recall.

17. A system for item recognition using augmented reality, the system comprising:
a medical storage device comprising:
a storage-device processor; and
a storage-device memory comprising an inventory database identifying:
items stored in the medical storage device; and
a location in the medical storage device of the items stored in the storage device; and
an augmented reality (AR) device communicatively coupled to the storage device, the AR device comprising:
a camera;
a display;
a memory comprising computer executable instructions; and
a processor configured to:
capture a plurality of images with the camera of the AR device, the plurality of images including an item;
extract image features from the plurality of images;
extract a plurality of words on the item from the plurality of images;
identify the item based on at least some of the image features and at least some of the plurality of words;
access at least one database of information relating to the item;
determine to deliver the item based on the information relating to the item;
receive a confirmation of delivery of the item from the storage device; and
update the inventory database upon confirmation of delivery of the item.

18. The system of claim 17, wherein the item comprises at least one of: a medication; a medical device; or a consumable.

19. The system of claim 18, wherein the medical storage device comprises an automated dispensing cabinet (ADC).

20. The system of claim 19, wherein the ADC comprises a plurality of drawers, each of which drawers comprising a plurality of compartments, wherein at least some of the items are stored each in a unique compartment.

21. The system of claim 20, wherein the processor is further configured to:
receive identification of the item from a user of the AR device;
determine a location of the item within the ADC; and
control the display to indicate the item location to the user.

22. The system of claim 21, wherein determining the location of the item within the ADC comprises:
querying the ADC for location information; and
receiving the location information from the inventory database of the ADC.

23. The system of claim 17, wherein the at least one database of information relating to the item comprises a medication database stored in memory accessible by the AR device via a communication network.

24. The system of claim 23, wherein determining to deliver the item is further based on patient information, wherein the patient information is associated with a recipient of the item.

25. The system of claim 24, wherein the processor is further configured to retrieve the patient information from a patient database.

26. The system of claim 25, wherein the patient database is stored in memory accessible by the AR device via the communication network.

27. The system of claim 26, wherein determining to deliver the item based on the information relating to the item further comprises determining that the item is not expired.

28. The system of claim 27, wherein determining to deliver the item based on the information relating to the item further comprises determining that a lot number of the item is not associated with a recall.
